(19) United States
(12) Patent Application Publication
(10) Pub. No.: US 2024/0092732 A1
(43) Pub. Date: Mar. 21, 2024

(54) PROCESS FOR PRODUCING TAURINE

(71) Applicant: Vitaworks IP, LLC, North Brunswick, NJ (US)

(72) Inventor: Songzhou Hu, Princeton, NJ (US)

(21) Appl. No.: 18/213,384

(22) Filed: Jun. 23, 2023

Related U.S. Application Data
(60) Provisional application No. 63/404,928, filed on Sep. 8, 2022.

(51) Int. Cl. C07C 303/32

(52) U.S. Cl. CPC C07C 303/32

(57) ABSTRACT

The present invention is directed to an improved process for producing taurine from ammonium isethionate without generating or using any acid within the cyclic process as recited in the claims appended hereto.

### CROSS REFERENCE

This application claims the benefit under 35 U.S.C. § 119(e) from U.S. Provisional Application No. 63/404,928, filed on Sep. 8, 2022, the contents of which are incorporated by reference herein.

### FIELD OF THE INVENTION

This invention relates to an improved process for preparing taurine from ammonium isethionate without using or generating any acid and without producing any byproducts of inorganic salts.

### BACKGROUND OF THE INVENTION

Taurine can be referred to as 2-aminoethanesulfonic acid and is of the formula H₂NCH₂CH₂SO₃H. Taurine is an extremely useful compound because it per se has such pharmacological effects as detoxification effect, fatigue-relieving effect, and nourishing and tonifying effect. As a result, taurine finds wide applications as an essential ingredient for human and animal nutrition.

Although there are numerous synthetic methods to prepare taurine and related derivatives, only two methods have been used commercially to manufacture over 80,000 tons of taurine per year, starting from ethylene oxide (the EO process) and monoethanolamine (the MEA process). The EO process accounts for more than 90% of taurine produced.

According to the traditional EO process, EO is reacted with sodium bisulfite to yield sodium isethionate, which undergoes an ammonolysis reaction to yield a mixture of sodium taurinate, disodium ditaurinate, and trisodium tritaurinate. Neutralization with sulfuric acid results in a mixture of taurine, sodium ditaurinate, sodium tritaurinate, and sodium sulfate. The main reactions are shown in the following scheme:

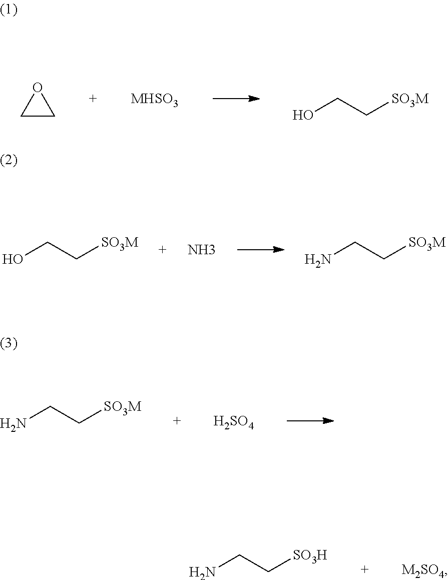

wherein M is an alkali. The alkali is lithium, sodium, potassium, or a mixture thereof.

Although the traditional EO process suffers two major problems, i.e., the generation of a large amount of waste mother liquor comprised of byproducts such as sodium ditaurinate and sodium tritaurinate and the coproduction of nearly equal amount of sodium sulfate, recent processes have ameliorated these problems to reach a nearly quantitative yield.

U.S. Pat. No. 9,428,450, reissued as U.S. RE48,238, U.S. RE48,333, and U.S. RE48,354, discloses the breakthrough finding that the byproducts of the EO process, i.e., sodium ditaurinate and sodium tritaurinate in the mother liquor solution, can be converted to sodium taurinate if they are converted to disodium ditaurinate and trisodium tritaurinate by reacting with at least equal molar amount of sodium hydroxide. This novel finding renders a cyclic process possible.

U.S. Pat. No. 9,428,451, reissued as U.S. RE48,392, describes a cyclic process for the production of taurine from sodium isethionate in a molar yield of at least 85% to a quantitative 100% by continuously converting the byproducts of sodium ditaurinate and sodium tritaurinate into taurine in each successive cycle.

U.S. Pat. No. 9,573,890, reissued as U.S. RE48,369, discloses a novel ammonolysis reaction of sodium isethionate in the presence of recycling mother liquor wherein the sodium ditaurinate and sodium tritaurinate are converted to disodium ditaurinate and trisodium tritaurinate to yield taurine in a yield of at least 85% to a quantitative 100% in the ammonolysis reaction.

U.S. Pat. No. 8,609,890 discloses a cyclic process by using isethionic acid or sulfur dioxide to neutralize alkali taurinate to produce taurine and to regenerate alkali isethionate.

U.S. Pat. No. 9,108,907 further discloses a process of using isethionic acid prepared from ethanol to neutralize alkali taurinate to regenerate alkali isethionate. The aim is to reduce or eliminate the use of sulfuric acid as an acid agent in the production of taurine.

U.S. Pat. No. 9,061,976 demonstrates an integrated production scheme by using sulfur dioxide or sulfurous acid as an acid and by converting the byproducts of the ammonolysis reaction, alkali ditaurinate and alkali tritaurinate, to alkali taurinate. The overall production yield is increased to greater than 90% and the alkali sulfate is eliminated from the production process. One drawback of this process is the use of gaseous sulfur dioxide, which imparts a slight smell on the product. Another significant drawback is that the taurine product from this process may contain trace amount of alkali sulfite which could be an allergen for certain people.

U.S. Pat. No. 10,071,955 discloses a cyclic process to produce taurine by using ion-exchange resin to neutralize sodium taurinate to taurine and then using sulfurous acid to regenerate the spent ion exchange resin to recover sodium bisulfite, which is subsequently reacted with ethylene oxide to produce sodium isethionate. In the cyclic process, the mother liquor after taurine crystallization is combined with sodium isethionate and subjected to an ammonolysis in the presence of sodium hydroxide to achieve a yield of more than 90%. One drawback of the process is the use of poisonous and obnoxious sulfurous acid. Another drawback of the process is the production of a dilute solution of taurine and a dilute solution of sodium bisulfite and the generation of large amount of waste water in the washing cycles of resin.

U.S. Pat. No. 9,593,076 improves the cyclic process disclosed in U.S. Pat. No. 8,609,890 for producing taurine from isethionic acid in an overall yield of greater than 90% to nearly quantitative, while generating no inorganic salt as byproduct. Similarly, CN 106008280A describes the use of isethionic acid to neutralize sodium taurinate and to regenerate sodium isethionate. However, the starting material, isethionic acid, is difficult to obtain commercially and is produced by a costly process of bipolar membrane electrodialysis of alkali isethionate.

U.S. Pat. Nos. 9,850,200 and 9,994,517 disclose a process for producing taurine by using an ammonium salt to react with alkali taurinate to yield taurine. In particular, ammonium bisulfite, ammonium sulfite, or their mixture is used to produce taurine and to regenerate a mixture of alkali bisulfite and alkali sulfite.

U.S. Pat. Nos. 9,745,258; 9,815,778; and 9,926,265 disclose a novel cyclic process that replaces acids, i.e., sulfuric acid, sulfurous acid, sulfur dioxide, and isethionic acid, with a neutral salt of ammonium isethionate to react with sodium taurinate to produce ammonium taurinate and to regenerate sodium isethionate. The ammonium taurinate is decomposed upon heating to taurine and ammonia, which is recovered for the ammonolysis reaction. The regenerated sodium isethionate and recovered ammonia are then combined to produce sodium taurinate, which is further reacted with ammonium isethionate to complete the cyclic process. The cyclic process according to this novel invention can reach a yield of nearly 100%. Since ammonium isethionate can be economically and conveniently prepared from ethylene oxide and ammonium bisulfite, this cyclic process becomes the most efficient and most economical. Moreover, this process yields taurine of exceptional quality. It is a foregone conclusion that this cyclic process will dominate the production of taurine in the future. The main reactions involved in this cyclic process can be described as follows:

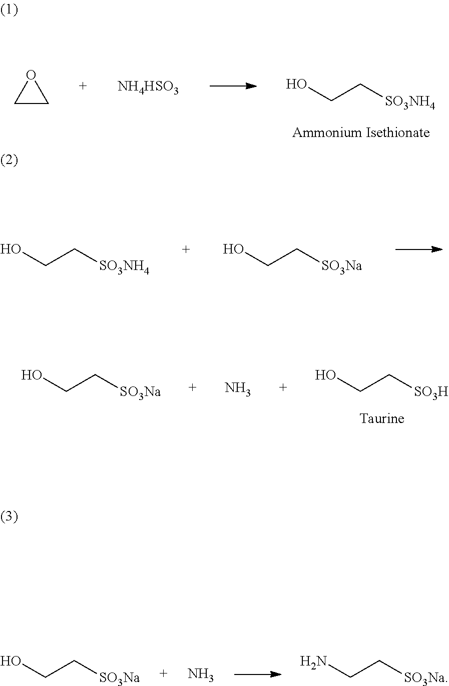

In the cyclic process disclosed in U.S. Pat. Nos. 9,745,258; 9,815,778; and 9,926,265, alkali hydroxide is needed and used for the ammonolysis of alkali isethionate in the recycling mother liquor solution. The alkali hydroxide is used in the process for two purposes. First, alkali hydroxide is needed as a catalyst for the ammonolysis of alkali isethionate. Second, alkali hydroxide is required to deprotonate taurine, alkali ditaurinate, and alkali tritaurinate, in the recycling mother liquor, to alkali taurinate, dialkali ditaurinate, and trialkali tritaurinate, respectively. Hence, the amount of alkali hydroxide is at least equal to the total molar amount of taurinates, including taurine, sodium ditaurinate, and sodium tritaurinate, in the recycling mother solution to achieve a nearly quantitative yield.

If external alkali hydroxide is introduced into the cyclic process, additional ammonium isethionate is required to neutralize the strong base. The result is an increased accumulation of alkali in each recycling stage. To ameliorate this technical problem, U.S. Pat. No. 9,815,778 discloses a process to generate the alkali hydroxide by splitting a mixture of alkali isethionate and alkali ditaurinate in the mother liquor into an acidic component, a mixture of isethionic acid and ditaurine, and an alkali hydroxide component, by using bipolar membrane electrodialysis. The mixed acidic solution of isethionic acid and ditaurine is used as an acid to neutralize alkali taurinate after the ammonolysis, while alkali hydroxide is used as a catalyst for the ammonolysis reaction.

However, there is a distinct disadvantage in the production and use of acids, since these mixed acids are known to be corrosive to process equipment made of stainless steel. As a result, the process is complicated and corrosion resistant equipment must be used in some parts of the production plant.

It is the object of the present invention to overcome this inherent disadvantage of the cyclic process and to disclose an improved process wherein no acid is generated or used throughout the entire cyclic process.

### SUMMARY OF THE INVENTION

The present invention is directed to an improved process for producing taurine from ammonium isethionate without generating or using any acid within the cyclic process as recited in the claims appended hereto.

The invention is accomplished by using bipolar membrane electrodialysis to produce a solution of alkali hydroxide from a solution comprised of alkali taurinate. A neutral solution of taurine or a basic solution of taurine and alkali taurinate, produced during the bipolar membrane electrodialysis, is further processed to yield taurine. The alkali sodium hydroxide solution generated from the bipolar membrane electrodialysis of alkali taurinate is used as a catalyst in the improved process to achieve a nearly quantitative yield of taurine from ammonium isethionate.

### BRIEF DESCRIPTION OF THE DRAWINGS

FIG. 1 illustrates one embodiment of the improved process for producing taurine from ammonium isethionate by generating alkali hydroxide from alkali taurinate through the use of bipolar membrane electrodialysis.

FIG. 2 illustrates another embodiment of the improved process for producing taurine from ammonium isethionate by generating alkali hydroxide from alkali taurinate through the use of bipolar membrane electrodialysis.

### DETAILED DESCRIPTION OF THE INVENTION

The present invention relates to an improved process for the production of taurine from ammonium isethionate in a high overall yield of greater than 90% to nearly quantitative without generating any inorganic salt as byproduct and without generating or using any acid in the process.

In the process according to the present invention, ammonium isethionate can be efficiently and economically produced by reacting ethylene oxide with ammonium bisulfite according to the following equation:

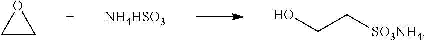

Ammonium isethionate, produced in a solution, can be used directly for the production of taurine. Preferably, ammonium isethionate is purified by concentrating the solution to obtain crystalline materials. When solid ammonium isethionate is used in the production of taurine, the quality of taurine produced is improved and almost no purge of mother liquor is required from the cyclic process.

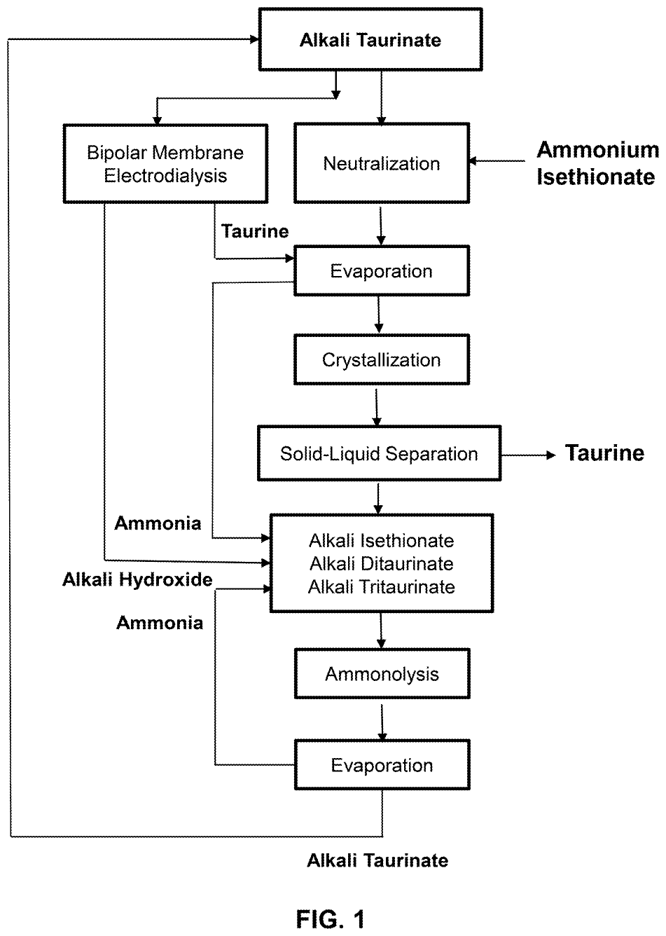

FIG. 1 illustrates one embodiment of the improved process for producing taurine from ammonium isethionate by generating alkali hydroxide from alkali taurinate through the use of bipolar membrane electrodialysis. The process according to the present invention starts with a solution comprised of alkali taurinate, which can be produced by methods known in the art, for example, by the ammonolysis of alkali isethionate or alkali isethionate in the recycling mother liquor.

The bipolar membrane electrodialysis can be carried out in any suitable electrodialyzer equipped with two electrodes, bipolar membrane, cation exchange membrane, and anion exchange membrane. Two cell configurations can be used for the bipolar membrane electrodialysis of alkali taurinate to produce alkali hydroxide. The first cell configuration is composed of three chambers for taurine, alkali taurinate, and alkali hydroxide, respectively. The membrane is arranged in the order of bipolar membrane, anion exchange membrane, cation exchange membrane, and bipolar membrane. In this cell configuration, a solution of taurine and a solution of alkali hydroxide are produced from a solution of alkali taurinate, respectively. The second cell configuration is composed of two chambers for alkali hydroxide and taurine-alkali taurinate. In this second cell configuration, the membrane is arranged in the order of bipolar membrane, cation exchange membrane, and bipolar membrane. In this second cell configuration, a solution of alkali hydroxide is produced and a mixed solution of taurine and alkali taurinate is obtained at the same time. Preferably, the second cell configuration is used to produce a solution of alkali hydroxide and a solution of taurine or a mixed solution of taurine and alkali taurinate.

The solution of alkali taurinate produced by the ammonolysis of alkali isethionate contains not only alkali taurinate, but other components such as dialkali ditaurinate, trialkali tritaurinate, and residual unreacted alkali isethionate. The presence of these other components will not affect the process, but requires strict monitoring and controlling of process parameters, in particular, the acidity of the solution to ensure the solution stays in the range from neutral to basic.

In the process according to FIG. 1, the solution of alkali taurinate is divided into at least two parts, preferably two parts. One part of the solution of alkali taurinate is passed through a bipolar membrane electrodialysis to produce a solution of taurine and a solution of alkali hydroxide. Before the solution of alkali taurinate enters the bipolar membrane electrodialysis, it is important to adjust the concentration of alkali taurinate, preferably to a concentration under 15% by weight, more preferably under 10%, most preferably under 8%. If the concentration of alkali taurinate is too high, after alkali taurinate is converted to taurine, taurine can precipitate from the solution to clog the membrane units.

When the solution of alkali taurinate produced by an ammonolysis of alkali isethionate undergoes exhaustive bipolar membrane electrodialysis, a strongly acidic solution is obtained. This surprising finding is due to the formation of isethionic acid, ditaurine, and tritaurine from unreacted alkali isethionate, dialkali ditaurinate, and trialkali tritaurinate, respectively. Since the acidic solution corrodes stainless process equipment and discolors the end product of taurine, it becomes necessary to control the bipolar membrane electrodialysis to the extent that the pH of the resultant taurine solution is at least greater than 4, preferably greater than 5, more preferably greater than 6, most preferably greater than 7. The taurine solution thus produced with a pH greater than 4 is not corrosive to process equipment made of stainless steel.

To maintain and control the pH of taurine solution, the size of the bipolar membrane electrodialysis unit and the amount of alkali taurinates entering the bipolar membrane electrodialysis unit may be adjusted accordingly.

The taurine solution after the bipolar membrane electrodialysis may be concentrated to isolate crystalline taurine. Preferably, this solution is mixed with the other part of the solution comprised of alkali taurinate before or after the addition of ammonium isethionate to regenerate alkali isethionate and to form ammonium taurinate. The ammonium taurinate is then decomposed to taurine by heating and removing ammonia from the solution. The temperature for decomposing ammonium taurinate is from 75° C. to 150° C., preferably from 90 to 120° C., most preferably from 95 to 110° C. Removal of ammonia released from the decomposition of ammonium taurinate can be carried out under reduced, normal, or increased pressure.

The reaction of alkali taurinate formed in the ammonolysis stage with ammonium isethionate proceeds according to the following equations:

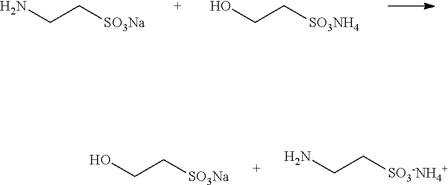

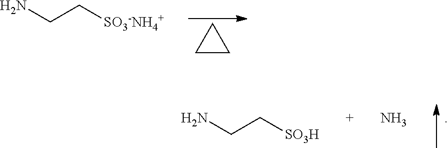

The amount of ammonium isethionate in relation to alkali taurinates including alkali taurinate, dialkali ditaurinate, and trialkali tritaurinate in the ammonolysis solution can be from 0.1 to 10, on the molar basis. Preferably, the molar ratio is from 0.5 to 1.5, more preferably from 0.9 to 1.1, and most preferably from 0.95 to 1.05. When the ratio is lower than the equivalent, the final pH after ammonia removal tends to be higher than 7 and more taurine will remain in the solution. When the ratio is greater than equivalent, the final pH is in the desirable range of 5 to 6, but additional alkali hydroxide will be consumed during the ammonolysis stage.

After alkali taurinates are reacted with ammonium isethionate and ammonia is removed from the solution by heating, alkali taurinate, dialkali ditaurinate, and trialkali tritaurinate are converted to their neutral form of taurine, alkali ditaurinate, and dialkali tritaurinate, respectively. During the reaction, alkali taurinate is reacted with ammonium isethionate to regenerate alkali isethionate and to form ammonium taurinate. The ammonium taurinate is then decomposed by heating to taurine and ammonia, which is removed from the solution and recovered for use.

The mother liquor solution after the separation of taurine is composed of regenerated alkali isethionate and other components, including, but not limited to, residual taurine, unreacted alkali isethionate, alkali ditaurinate, and alkali tritaurinate, all of which can be converted to alkali taurinate under appropriate reaction conditions during the recycling of mother liquor solution.

To the mother liquor solution is then added alkali hydroxide produced in the bipolar membrane electrodialysis step. Optionally, this mixed solution is concentrated, followed by adding excess ammonia, and the solution is then subject to an ammonolysis reaction to form a solution comprised of alkali taurinate, along with dialkali ditaurinate and trialkali tritaurinate. Part of this solution of alkali taurinate can be used, before or after the removal of excess ammonia, for the reaction with ammonium isethionate. Part of this solution after the removal of excess ammonia can be used to produce a solution of alkali hydroxide by bipolar membrane electrodialysis.

There is no limit to the amount of the solution of alkali taurinate that passes through the bipolar membrane electrodialysis relative to the total amount of the solution of alkali taurinate to produce alkali hydroxide. However, if less amount of alkali hydroxide is generated in the bipolar membrane electrodialysis stage from alkali taurinate, ammonolysis of alkali isethionate in the recycling mother liquor will not form alkali taurinate in a desirable yield to allow a steady recycling of the mother liquor. If too much alkali hydroxide is generated in the bipolar membrane electrodialysis stage of alkali taurinate, the process becomes less efficient, but a high yield of alkali taurinate can be attained and a steady state of recycling can be readily achieved.

It has been found that if the amount of the solution of alkali taurinate that passes through the bipolar membrane electrodialysis is less than 15%, the overall yield of taurine is drastically depressed. The mother liquor solution could not be effectively recycled to produce taurine. If the amount of the solution of alkali taurinate that passes through the bipolar membrane electrodialysis is more than 35%, the yield of taurine is high, but the efficiency of process is reduced. The finding of this preferable range is surprising and unexpected.

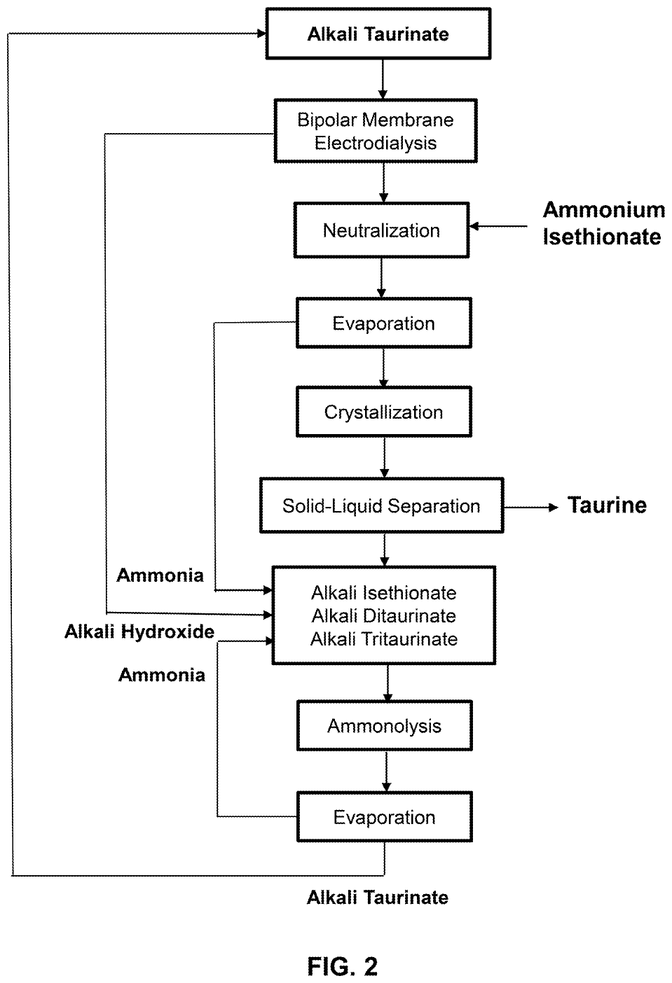

FIG. 2 illustrates another embodiment of the cyclic process for producing taurine from ammonium isethionate by generating alkali hydroxide from alkali taurinates through the use of bipolar membrane electrodialysis, wherein the alkali taurinates include alkali taurinate, dialkali ditaurinate, and trialkali tritaurinate. In this cyclic process, all the solution of alkali taurinates is passed through a bipolar membrane electrodialysis unit to produce a solution of alkali hydroxide and a solution comprising alkali taurinates.

There is no limit as to the amount of alkali taurinates that is converted to alkali hydroxide and taurine after passing through the bipolar membrane electrodialysis relative to the total amount of alkali taurinates in the solution. The amount can be determined by the amount of alkali hydroxide needed for the ammonolysis of recycling mother liquor solution. The amount of alkali hydroxide is at least equal to the total molar amount of taurinates in the recycling mother liquor solution, wherein taurinates include residual taurine, alkali ditaurinate, and alkali tritaurinate. Conveniently, a sufficient amount of alkali hydroxide is added to the recycling mother to a pH of preferably at least 9.5, more preferably at least 11, most preferably at least 13. At a pH greater than 11, all taurine components are fully deprotonated to taurinates for the ammonolysis reaction.

It has been found that the amount of alkali hydroxide is preferably in the range of 15% to 35% on the molar basis of total alkali taurinates in the solution of ammonolysis. If a lesser amount of alkali hydroxide is generated in the bipolar membrane electrodialysis stage, ammonolysis of alkali isethionate in the recycling mother liquor will not form alkali taurinate in a desirable yield to allow a steady recycling of the mother liquor. If too much alkali hydroxide is generated in the bipolar membrane electrodialysis stage, the process becomes less efficient, but a high yield of alkali taurinate can be attained and a steady state of recycling can be achieved.

After the solution of alkali taurinate is passed through the bipolar membrane electrodialysis to produce a solution of alkali hydroxide, a solution mixture of taurine and alkali taurinate is obtained. Ammonium isethionate is then added to this mixture to regenerate alkali isethionate and ammonium taurinate. After ammonium taurinate is decomposed and ammonia is removed, taurine is obtained by crystallization and solid-liquid separation to yield a mother liquor solution.

The mother liquor solution after the separation of taurine is composed of regenerated alkali isethionate and many other components. These components include, but not limited to, residual taurine, unreacted alkali isethionate, alkali ditaurinate, and alkali tritaurinate, all of which can be converted to alkali taurinate under disclosed reaction conditions during the recycling of the mother liquor.

To the mother liquor solution is then added alkali hydroxide produced in the bipolar membrane electrodialysis step, optionally, this mixed solution is concentrated, followed by adding excess ammonia, and the solution is then subject to an ammonolysis reaction to form a solution comprised of alkali taurinate, along with dialkali ditaurinate and trialkali tritaurinate. Before or after the removal of excess ammonia, the solution of alkali taurinate can be used to generate a solution of alkali hydroxide and then reacted with ammonium isethionate to produce taurine.

It is noted that the improved process according to the present invention has no step that requires the generation or use of any acid for the production of taurine from ammonium isethionate. Alkali taurinates are only partially converted to taurine and alkali hydroxide in the improved process according to present invention. As a result, the improved process according to the present invention eliminates the use of corrosion resistant equipment and ameliorates disadvantages in the process disclosed in prior art references.

It is further noted that the process according to present invention does not impose any requirement for the quality of alkali hydroxide generated in the bipolar membrane electrodialysis, as long as the quantity of alkali hydroxide in the solution is sufficient to deprotonate all taurinates in the recycling mother liquor to achieve the ammonolysis of alkali isethionate in a desired yield. Any contamination in the solution of alkali hydroxide from components that are present in the solution of alkali taurinate, such as alkali taurinate, alkali ditaurinate, alkali tritaurinate, or alkali isethionate, will not hinder its use as an effective catalyst for the ammonolysis of alkali isethionate. Alkali hydroxide in the process according to the present invention only serves as a basic agent and any impurities present in the alkali hydroxide due to any imperfection in the bipolar membrane will not react with ethylene oxide to form byproducts. This inherent advantage of the improved process according to present invention effectively prolongs the useful lifespan of the bipolar membrane and thus greatly reduces production cost.

The process according to the present invention can produce taurine in a molar yield of at least 85%, preferably at least 90%, more preferably at least 95%, most preferably quantitatively at 100% on the basis of ammonium isethionate.

The process according to the present invention can produce taurine that does not contain any salt of sulfate or chloride as an impurity of inorganic salts.

The process according to the present invention can be carried out discontinuously, semi-continuously, or continuously.

### EXAMPLES

The following examples will illustrate the practice of this invention but are not intended to limit its scope.

### Example 1

To an autoclave were added 600 mL of 25% ammonium hydroxide, 148.2 g of sodium isethionate, and 1 g of sodium hydroxide. After the autoclave was heated to 220° C. for 3 hours under autogenous pressure, the autoclave was cooled to room temperature and the solution was distilled to remove excess ammonia to obtain a solution comprising sodium taurinate, which was diluted to 500 mL with water.

The solution of sodium taurinate was circulated in a two-chamber bipolar membrane electrodialysis setup with the following membranes: Fumasep FBM-PK and Fumasep FKB-PK-130. After the concentration of sodium hydroxide in 300 mL of solution reached about 4%, the bipolar membrane electrodialysis was stopped. To the solution of sodium taurinate after bipolar electrodialysis was added 94.0 g of ammonium isethionate. After the solution was then heated to boiling to expel ammonia and concentrated to 350 mL, the solution was cooled to 10° C. to crystallize taurine, which was separated by filtration and washing with ice-cold water. The mother liquor solution was concentrated to about 250 g and cooled to room temperature to obtain additional taurine. After drying, 86.0 g of taurine was obtained in a molar yield of 68.8% on the basis of sodium isethionate. The yield was 91.7% on the basis of ammonium isethionate.

### Example 2

The mother liquor solution of Example 1 was then combined with the solution of sodium hydroxide generated from the bipolar membrane electrodialysis in Example 1 and concentrated to about 280 mL and mixed with 600 mL of 25% ammonium hydroxide. After the autoclave was heated to 220° C. for 3 hours under autogenous pressure, the autoclave was cooled to room temperature and the solution was distilled to remove excess ammonia to obtain a solution comprising sodium taurinate, which was diluted to 500 mL with water.

The solution of sodium taurinate was circulated in the same two-chamber bipolar membrane electrodialysis setup as used in Example 1. After the concentration of sodium hydroxide in 300 mL of solution reached about 4%, the bipolar membrane electrodialysis was stopped. To the solution of sodium taurinate after bipolar electrodialysis was added 94.0 g of ammonium isethionate. After the solution was then heated to boiling to expel ammonia and concentrated to 350 mL, the solution was cooled to 10° C. to crystallize taurine, which was separated by filtration and washing with ice-cold water. The mother liquor solution was concentrated to about 250 g and cooled to room temperature to obtain additional taurine. After drying, 89.0 g of taurine was obtained in a molar yield of 94.9% on the basis of ammonium isethionate.

### Example 3

To an autoclave were added 600 mL of 25% ammonium hydroxide, 148.2 g of sodium isethionate, and 1 g of sodium hydroxide. After the autoclave was heated to 220° C. for 3 hours under autogenous pressure, the autoclave was cooled to room temperature and the solution was distilled to remove excess ammonia to obtain a solution comprising sodium taurinate, which was diluted to 500 mL with water.

125 mL of the solution comprising sodium taurinate was diluted to 400 mL with water and circulated in a two-chamber bipolar membrane electrodialysis setup as used in Example 1. After the pH of the sodium taurinate chamber became neutral, the bipolar membrane electrodialysis was stopped.

To the remaining solution of sodium taurinate was added 94.0 g of ammonium isethionate. The solution was combined with the taurine solution after bipolar membrane electrodialysis. After the solution was heated to boiling to expel ammonia and concentrated to 350 mL, the solution was cooled to 10° C. to crystallize taurine, which was separated by filtration and washing with ice-cold water. The mother liquor solution was concentrated to about 250 g and cooled to room temperature to obtain additional taurine. After drying, 86.5 g of taurine was obtained in a molar yield of 69.2% on the basis of sodium isethionate. The yield was 92.3% on the basis of ammonium isethionate.

### Example 4

The mother liquor solution of Example 3 was then combined with the solution of sodium hydroxide generated from the bipolar membrane electrodialysis in Example 3 and concentrated to about 280 mL and mixed with 600 mL of 25% ammonium hydroxide. After the autoclave was heated to 220° C. for 3 hours under autogenous pressure, the autoclave was cooled to room temperature and the solution was distilled to remove excess ammonia to obtain a solution comprising sodium taurinate, which was diluted to 500 mL with water.

125 mL of the solution comprising sodium taurinate was diluted to 400 mL with water and circulated in a two-chamber bipolar membrane electrodialysis setup as used in Example 1 to produce a solution of taurine and a solution of sodium hydroxide. After the pH of the sodium taurinate chamber became neutral, the bipolar membrane electrodialysis was stopped.

To the remaining solution of sodium taurinate was added 94.0 g of ammonium isethionate. The solution was combined with the taurine solution generated from the bipolar membrane electrodialysis of sodium taurinate. After the solution was heated to boiling to expel ammonia and concentrated to 350 mL, the solution was cooled to 10° C. to crystallize taurine, which was separated by filtration and washing with ice-cold water. The mother liquor solution was concentrated to about 250 g and cooled room temperature to obtain additional taurine. After drying, 90.3 g of taurine was obtained in a molar yield of 96.3% on the basis of ammonium isethionate.

It will be understood that the foregoing examples, explanation, and drawings are for illustrative purposes only and that in view of the instant disclosure various modifications of the present invention will be self-evident to those skilled in the art. Such modifications are to be included within the spirit and purview of this application and the scope of the appended claims.

What is claimed is:

1. A process for producing taurine, comprising:
(a) adding excess ammonia and a catalytic amount of alkali hydroxide to a mother liquor solution comprising alkali isethionate; wherein the catalytic amount of alkali hydroxide is produced in a bipolar membrane electrodialysis of step (c) and wherein the mother liquor solution comprising alkali isethionate is produced in step (d);
(b) subjecting the solution of step (a) to an ammonolysis, and optionally removing excess ammonia, to yield a solution comprising alkali taurinate;
(c) passing the solution comprising alkali taurinate of step (b) through a bipolar membrane electrodialysis to produce a catalytic amount of alkali hydroxide and a solution comprising taurine and alkali taurinate; and
(d) adding ammonium isethionate to the solution comprising taurine and alkali taurinate of step (c) to obtain taurine and a mother liquor solution comprising alkali isethionate.

2. The process according to claim 1, wherein the bipolar membrane electrodialysis is carried out in a three-chamber configuration of taurine, alkali taurinate, and alkali hydroxide, respectively.

3. The process according to claim 1, wherein the bipolar membrane electrodialysis is carried out in a two-chamber configuration of alkali hydroxide and alkali taurinate.

4. The process according to claim 1, wherein the catalytic amount of alkali hydroxide produced in the bipolar membrane electrodialysis of step (c) is at least equal to the total molar amount of taurinates in the mother liquor solution of step (a).

5. The process according to claim 1, wherein the catalytic amount of alkali hydroxide produced in the bipolar membrane electrodialysis of step (c) is from 15% to 35% of the molar amount of alkali isethionate in the mother liquor solution.

6. The process according to claim 1, wherein the molar yield of taurine is greater than 90% on the basis of ammonium isethionate.

7. The process according to claim 1, wherein the taurine does not contain salt of sulfate or chloride as impurity.

8. A process for producing taurine, comprising:
(a) adding excess ammonia and a catalytic amount of alkali hydroxide to a mother liquor solution comprising alkali isethionate; wherein the catalytic amount of alkali hydroxide is produced in a bipolar membrane electrodialysis of step (c) and wherein the mother liquor solution is produced in step (d);
(b) subjecting the solution of step (a) to an ammonolysis, and optionally removing excess ammonia, to yield a solution comprising alkali taurinate;
(c) passing part of the solution comprising alkali taurinate of step (b) through a bipolar membrane electrodialysis to produce a catalytic amount of alkali hydroxide and a solution comprising taurine; and
(d) adding ammonium isethionate to the other part of the solution comprising alkali taurinate of step (b) to obtain taurine and a mother liquor solution comprising alkali isethionate.

9. The process according to claim 8, wherein the bipolar membrane electrodialysis is carried out in a three-chamber configuration of taurine, alkali taurinate, and alkali hydroxide, respectively.

10. The process according to claim 8, wherein the bipolar membrane electrodialysis is carried out in a two-chamber configuration of alkali hydroxide and alkali taurinate.

11. The process according to claim 8, wherein the amount of the solution comprising alkali taurinate passing through the bipolar membrane electrodialysis is from 15% to 35% of the solution comprising alkali taurinate.

12. The process according to claim 8, wherein the solution comprising taurine of step (c) has a pH of greater than 4.

13. The process according to claim 8, wherein the catalytic amount of alkali hydroxide produced in the bipolar membrane electrodialysis of step (c) is at least equal to the total molar amount of taurinates in the mother liquor solution of step (a).

14. The process according to claim 8, wherein the catalytic amount of alkali hydroxide produced in the bipolar membrane electrodialysis of step (c) is from 15% to 35% of the molar amount of alkali isethionate in the mother liquor solution.

15. The process according to claim 8, wherein the molar yield of taurine is greater than 90% on the basis of ammonium isethionate.
